(19) United States
(12) Patent Application Publication
(10) Pub. No.: US 2026/0355992 A1
(43) Pub. Date: Nov. 12, 2026

(54) SHAVER

(71) Applicant: BIC Violex Single Member S.A., Anoixi (GR)

(72) Inventor: Nikolaos CHRYSANTHAKOPOULOS, Anoixi (GR)

(73) Assignee: BIC Violex Single Member S.A., Anoixi (GR)

(21) Appl. No.: 19/134,963

(22) PCT Filed: Dec. 20, 2023
(86) PCT No.: PCT/EP2023/086796

§ 371 Date: Jun. 2, 2025

(30) Foreign Application Priority Data
Dec. 22, 2022 (EP) 22215798.4

(51) Int. Cl. B26B 21/40; B26B 21/22; B26B 21/48; G01N 1/02; G01N 1/10; G01N 1/14; G01N 33/487

(52) U.S. Cl. CPC B26B 21/4056; B26B 21/22; B26B 21/4025; B26B 21/48; G01N 1/10; G01N 33/48792; G01N 2001/028; G01N 2001/149

(57) ABSTRACT

A shaving head for a shaver including an absorbent area configured to receive a body sample extracted from a user in a shaving stroke of the shaver, wherein the absorbent area is configured to be coupled to a biosensor unit of the shaver. A shaver including a handle and the shaving head. A health monitoring system including the shaver and an electronic device configured to be communicatively coupled to the shaver. A computer-implemented method for health monitoring, including operating a biosensor unit in the shaver to analyze a body sample extracted from a user in a shaving stroke of the shaver, thereby generating an analysis result.

### CROSS REFERENCE TO RELATED APPLICATIONS

This application claims benefit from European patent application EP 22215798.4 filed on 22 Dec. 2022, its content being incorporated herein by reference.

### TECHNICAL FIELD

This specification relates to a shaving head, a shaver, a health monitoring system and a computer-implemented method for health monitoring.

### BACKGROUND

Health monitoring and tracking is offered by plenty wearable medical devices that are configured to collect health metrics data and provide early diagnostics. Such devices may comprise one or more biosensors.

A biosensor may be an analytical device that combines a biological element with a physicochemical detector in order to detect a chemical substance and/or measure a quantitative degree thereof. The sensitive biological element, e.g. tissue, microorganisms, organelles, cell receptors, enzymes, antibodies, nucleic acids, etc., may be a biologically derived material or biomimetic component that interacts with, binds with, or recognizes the analyte under study. Low-cost electronic biosensors made of semiconducting polymer, capable of detecting quickly and with high sensitivity, a wide range of health conditions from body fluids, have been developed.

Such biosensors may be configured to measure the amount of critical metabolites, such as lactate or glucose, that are present in sweat, tears, saliva or blood, and, when incorporated into a diagnostic device, may allow health conditions to be monitored quickly, cheaply and accurately. Recently, the design of such biosensors has been simplified, thereby opening up a wide range of new possibilities for health monitoring down to the cellular level. For example, a low-cost biosensor may measure levels of lactate, which is useful for example in fitness applications. However, such a biosensor may be easily modified to detect e.g. glucose or cholesterol by incorporating the appropriate enzymes.

To build the biosensor, newly-synthesized polymers may be used that act as a molecular wire, directly accepting the electrons produced during electrochemical reactions. When the material comes into contact with a liquid such as sweat, tears or blood, it may absorb ions and swells, becoming merged with the liquid. This has been found to lead to significantly higher sensitivity compared to traditional sensors made of metal electrodes.

Additionally, when the biosensors are incorporated into more complex circuits, such as transistors, the signal may be amplified and respond to tiny fluctuations in metabolite concentration, despite the tiny size of the devices.

Since biosensors do not necessarily consist of metals such as gold or platinum, they may be manufactured at a lower cost and may be easily incorporated in flexible and stretchable substrates, enabling their implementation in wearable or implantable sensing applications.

Microfluidics for real time diagnostics is known. A microfluidic chip may be a pattern of microchannels, molded or engraved. Such a network of microchannels incorporated into the microfluidic chip may be linked to the macro-environment by several holes of different dimensions hollowed out through the chip. It is through these pathways that fluids are injected into and evacuated from the microfluidic chip. Fluids may be directed, mixed, separated or manipulated to attain multiplexing, automation, and/or high-throughput systems. The microchannels network design has typically be precisely elaborated to achieve the desired features (lab-on-a-chip, detection of pathogens, electrophoresis, DNA analysis etc.).

Microfluidic devices may exploit the physical and chemical properties of liquids and gases at a microscale. Microfluidic devices offer several benefits over conventionally sized systems. Microfluidics allow the analysis and use of less volume of samples, chemicals and reagents reducing the global fees of applications. Many operations may be executed at the same time thanks to their compact size, shortening the time of experiment. They are also known to offer an excellent data quality and substantial parameter control which allows process automation while preserving the performances. They have the capacity to both process and analyze samples with minor sample handling.

Microfluidics may feature faster reaction time, enhanced analytical sensitivity, enhanced temperature control, portability, easier automation and parallelization, integration of lab routines in one device (lab-on-a-chip). It may be comparatively cheap as it does not involve the use of various costly equipment. Great advancements on microfluidics and lab-on-a-chip technology for the microfiltering and analysis of sweat, blood and other body fluids have been achieved. In one example, a magnetically driven rotary microfilter has been fabricated that may be used to filter particles inside a microfluidic device. The tiny turning filter has been made by creating a magnetic material that could be used with a very precise 3D printing technique known as two-photon polymerization, wherein a liquid light-sensitive material known as photoresist is polymerized. Thanks to two-photon absorption, the polymerization may be done in a very precise manner, enabling fabrication of complex structures on the micron scale. To make the microfilter magnetic, magnetic nanoparticles were synthesized and mixed with a photoresist.

By changing the direction of external magnetic field, the microfilter may be remotely manipulated on demand to either filter certain-sized particles and/or to allow them all to pass.

To filter larger particles, a magnetic field perpendicular to the microchannel may be applied. After the filtering process is complete, the large particles may be released—i.e. the filter network may be reset—by applying a magnetic field that is parallel to the microchannel, which will rotate the microfilter by 90°. The filtering process may then be repeated as needed.

Electromagnetic coils are known to be produced by winding an electrical conductor such as a wire, into the shape of a coil, spiral, or helix. The shape and dimensions of a coil are designed to fulfill a particular purpose. Parameters such as inductance, resistance, and strength of the desired magnetic field greatly influence the design of a coil's shape and size. Micro coils may be very small, often requiring the use of a magnifying glass or microscope to see and work with them.

Electromagnetic coils may be used in applications where electric currents interact with magnetic fields, in devices such as inductors, electromagnets, transformers, and sensor coils. An electric current through the wire of the coil is known to generate a magnetic field.

Small scale electromagnets have been developed for small scale medical use. There exist simple, inexpensive designs that use soft ferromagnetic cores to expose ~2 cm³ working volumes to amplitudes of tens of kA/m at frequencies between 100 kHz and 1 MHz.

A variety of small, strong magnets has become commercially available. A method for producing magnets with a desktop 3D printer has been developed, thereby succeeding in integrating magnetic properties into various 3D printed parts, all without post-magnetization.

### SUMMARY

According to a first aspect, there is provided a shaving head for a shaver, wherein the shaving head comprises an absorbent area configured to receive a body sample extracted from a user in a shaving stroke of the shaver. The absorbent area is configured to be coupled to a biosensor unit of the shaver. The shaving head may or may not comprise a filter network configured to filter and/or drive the body sample or parts thereof from the absorbent area to the one or more sensors of the biosensor unit.

According to a second aspect, there is provided a shaver comprising a handle and the shaving head of the first aspect (or an embodiment thereof).

According to a third aspect, there is provided a health monitoring system comprising the shaver of the second aspect (or an embodiment thereof) and an electronic device configured to be communicatively coupled to the shaver.

According to a fourth aspect, there is provided a computer-implemented method for health monitoring. The method comprises operating a biosensor unit in a shaver to analyze a body sample extracted from a user in a shaving stroke of the shaver, thereby generating an analysis result.

According to a fifth aspect, there is provided a computer system configured to execute the computer-implemented method for health monitoring of the fourth aspect (or an embodiment thereof).

According to a sixth aspect, there is provided a computer program configured to execute the computer-implemented method for health monitoring of the fourth aspect (or an embodiment thereof).

According to a seventh aspect, there is provided a computer-readable medium or signal storing the computer program of the sixth aspect (or an embodiment thereof).

Dependent embodiments of the aforementioned aspects are given in the dependent claims/embodiments and explained in the following description, to which the reader should now refer.

Conventionally, health related testing may be performed through dedicated procedures using specific devices (other than a shaver) requiring from the user to spend effort, time and/or money. Many regular vital sign metrics or tests of chronic or acute illness biomarkers are usually performed in specialized points of care or rarely through special self-test kits. A user may neglect or forget to allocate time to perform a specific health metrics test regularly. Furthermore, blood (or urine) collection for health tests may usually be painful or uncomfortable.

Shaving action is a regular action performed by many women and men for most of their lives. The objective of a shaving action is to cut the hair portion extending over the skin surface. However, usually during hair cutting, dermaplaning—i.e. removal of cells or layers of the stratum corneum of the skin—and superficial skin nicks and cuts may occur, permitting skin cells, blood traces, interstitial fluid, and the like to come temporarily on skin surface. Also, regularly, sebaceous and sweat glands secrete sebum and sweat respectively on the skin surface.

The shaving head, the shaver, the health monitoring system and/or the method for health monitoring of the aforementioned aspects are directed to health monitoring during shaving. In other words, they serve to integrate regular health monitoring into the (daily) routine of shaving, thereby facilitating health monitoring for users. In fact, while interaction such as e.g. selecting and/or specifying a user-defined testing protocol is possible, any extra action by the user (beyond shaving) is not required.

When a user shaves a skin portion such as e.g. her/his (or another's) face or legs in one or more shaving strokes, blades of the shaver predominantly cut hair but also scratch skin thereby removing upper stratum corneum (SC) layers. In so doing, blade cutting action may expose on the skin body fluids and/or live skin cells, that are usually washed up. For example, micro nicks and/or cuts may result in minor blood spots and interstitial fluid. Furthermore, glands secrete sebum and sweat. Such body elements extracted from the user being shaved may be used as a body sample to be analyzed for health monitoring. For example, with each shaving stroke the absorbent area of the shaving head passes over the skin shaved by the blades action. The absorbent area absorbs any sweat, sebum, blood, interstitial fluid etc. found on the skin, thereby receiving the body sample to be analyzed. Subsequently, one or more microfluidic network filters may be employed in the shaving head (and/or in the handle of the shaver) to filter and/or drive the body sample to one or more biosensors of the biosensor unit of the shaver. The one or more biosensors may then analyze the body sample for specific health metrics (e.g. depending on a user-defined testing protocol). For example, the one or more biosensors may then generate a digital analysis result that may be presented via a user output interface of the (smart) shaver or be transmitted to a communicatively coupled electronic device such as e.g. a smart phone.

In embodiments, the shaving head may comprise a cover configured to cover or uncover (depending on the state) the absorbent area. For example, adjusting the cover to cover the absorbent area is useful when rinsing the shaving head, thereby preventing water from being absorbed by the absorbent area and flooding the one or more biosensors or e.g. the filter network leading thereto.

In embodiments, the shaving head and/or the handle of the shaver may comprise a filter network configured to filter and/or drive the body sample or parts thereof from the absorbent area to the one or more sensors of the biosensor unit. This filter network may be static or dynamically adjustable. In the latter case, the shaving head and/or the handle may comprise a filter element configured to adjust and/or reset the filter network. For example, the filter network may be a magnetically driven rotary microfilter that may be adjusted and/or reset via an external magnetic field. In adjusting the magnetic field appropriately, varying analyses for health monitoring may be performed depending on varying and/or user-defined testing protocols. In so doing, users may choose among different health metrics analyses for the same biosensing unit, depending on their needs.

In embodiments, the shaving head may comprise a heating element configured to heat up a skin portion of the user in and at the beginning of a shaving stroke. For example, this may be useful for increasing secretion of sebum and/or sweat, thereby enhancing health monitoring. Health monitoring (also) based on the secretion of sebum and/or sweat is beneficial as such secretion does not rely on skin injury during shaving.

In embodiments, one or more disposable biosensors and absorbent area may be implemented as an addon to a regular shaver.

### FIGURE DESCRIPTION

FIG. 1 illustrates an example embodiment of a shaver with a shaving head comprising an absorbent area configured to receive a body sample extracted from a user in a shaving stroke of the shaver.

FIG. 2 schematically illustrates a shaving head comprising an absorbent area configured to receive a body sample extracted from a user in a shaving stroke of the shaver.

FIG. 3 schematically illustrates a shaver configured to receive a body sample extracted from a user in a shaving stroke of the shaver.

FIG. 4 schematically illustrates a health monitoring system.

FIG. 5 schematically illustrates a computer-implemented method for health monitoring during shaving.

FIG. 6 illustrates an example scenario for health monitoring during shaving.

### DETAILED DESCRIPTION

The shaving head, the shaver, the health monitoring system and the computer-implemented method for health monitoring as disclosed in this specification are based on the idea to combine shaving with health monitoring.

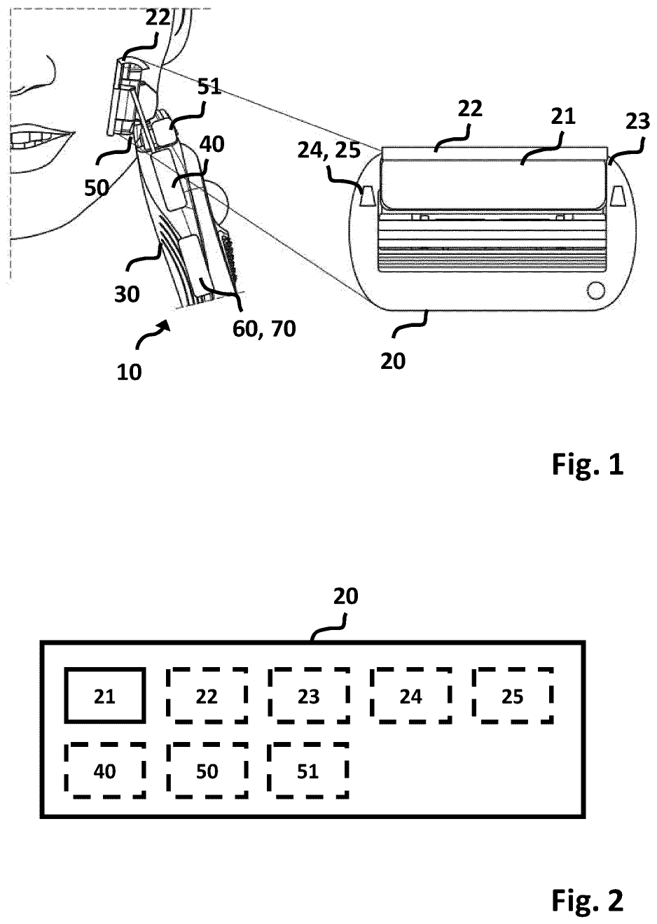

An example scenario may be as follows: A user gets her/his shaver to shave. The shaver, as for example displayed in FIG. 1, may contain a biosensor unit, or the user may insert a specific biosensor unit according to her/his needs. The user may select a specific testing protocol and perform a one or more shaving strokes. A shaving stroke may induce dermaplaning, skin exfoliation and/or micro skin cuts on the shaved area. As a result, skin cells, blood and/or interstitial fluid may appear on the skin surface after a shaving stroke. Furthermore, and independently of skin injuries, sebum and sweat may be secreted continuously on the skin. For example, secretion may be accelerated by the pressure of the shaving head and/or the action of a heating element of the shaving head. An absorbent area located, for example, at the front top area of the shaving head collects a body sample comprising skin cells and/or skin excretions. For example, a microfluidic network filter may collect the body sample or parts thereof from the absorbent area and filter and/or drive them to one or more biosensors of the biosensor unit. These one or more biosensors then analyze the body sample, thereby generating an analysis result. For example, the analysis result is communicated to the user visually and/or is transmitted e.g. through NFC to an electronic device such a smartphone. The electronic device may be configured to visualize the analysis result to the user e.g. via a software app. Another example scenario is given in FIG. 6.

There is disclosed a shaving head 20 for a shaver 10, wherein the shaving head 20 comprises an absorbent area 21 configured to receive a body sample extracted from a user in a shaving stroke of the shaver 10. The absorbent area 21 is configured to be coupled to a biosensor unit 40 of the shaver 10. The shaving head 20 may comprise one or more blades configured for shaving. A shaving head 20 is schematically illustrated in FIG. 2.

For example, the body sample may comprise blood. Alternatively, or in addition, the body sample may comprise an interstitial fluid. Alternatively, or in addition, the body sample may comprise sweat. Alternatively, or in addition, the body sample may comprise sebum. Alternatively, or in addition, the body sample may comprise cells such as e.g. skin cells. Alternatively, or in addition, the body sample may comprise cell fragments.

The absorbent area 21 may be an area made of porous material capable of absorbing the body sample such as e.g. the skin secretions found on the skin after the shaving action. The absorbent area may be a natural or artificial microfluidic porous material. The absorbent material may be hydrophilic or oleophilic. For example, the absorbent area may comprise one or more polymers of PS, PU, PMMA, PEGDA, PFA. Alternatively, or in addition, the absorbent area may comprise paper and/or cellulose. Alternatively, or in addition, the absorbent area may comprise a sponge. Alternatively, or in addition, the absorbent area may comprise a fabric. Alternatively, or in addition, the absorbent area may comprise a hydrogel. Alternatively, or in addition, the absorbent area may comprise a composite. Alternatively, or in addition, the absorbent area may comprise a ceramic. Alternatively, or in addition, the absorbent area may comprise a silicon. In any case, the pores of the absorbent area may be orderly distributed or may be randomly distributed along the rim surface. For example, the pore size may range from 30 μm to 300 μm or, more preferable, from 70 to 150 μm.

The one or more blades cutting the hair during a shaving stroke may be configured to carry away the cut hair, the water and the shaving preparation, exposing the skin. Due to e.g. the dermaplaning action of the blades, traces of blood, skin interstitial fluid and skin cells may be found on the skin, after or towards the end of a shaving stroke. Also, sebaceous and sweat glands, secrete sebum and sweat continuously on the skin surface and this secretion flow may be accelerated momentarily by the pressure of the shaving head or through a heating action of the shaver head. The absorbent area may be arranged on the shaving head so as to absorb the body sample exposed on the skin towards the end of the shaving stroke.

The absorbent area may be part of a replaceable insert (e.g. also comprising the filter network 50 and the biosensor unit 40) that may be mounted on the shaving head.

The shaving head 20 may comprise a filter network 50 configured to filter and/or drive the body sample or parts thereof from the absorbent area 21 to the one or more sensors of the biosensor unit 40 (which may or may not be in the shaving head 20). The filter network 50 may be a microfluidic network. The filter network 50 may be a magnetically driven rotary microfilter configured to be adjusted and/or reset via an external magnetic field (e.g. resulting from an alternating magnetic field module).

The shaving head 20 may comprise a cover 22 configured to cover the absorbent area 21 in a first state and to uncover the absorbent area 21 in a second state. The shaving head 20 may comprise a cover mechanism configured to close and/or open the cover 22. The cover mechanism may comprise a cover actuator.

For example, the absorbent area may be covered by a movable lid, when not in touch with the skin, to minimize contamination and/or water absorption. The cover may be composed by one, two or more parts. The cover parts may translate, rotate and/or retreat to uncover the absorbent area when in proximity or in contact with the skin (as sensed by a contact senor unit 24 and/or a proximity sensor unit 25).

The actuation of the cover may be manual (in which case no cover mechanism exists), such as e.g. by user action. In other examples, the cover mechanism may be mechanical, such as e.g. through a lever mechanism activated by the contact of the skin to the shaving head through one or more protrusions pressed and/or rotated by the skin. In other examples, the cover mechanism may be electromechanical, such as e.g. through a sensor detecting the contact and/or the approach of the shaving head with the skin and sending a signal (based e.g. on the start signal, the contact signal and/or the proximity signal) to move the cover to its open position.

The opening of the cover may drive its parts to a non-obstructive position for the shaving action, over the top and back of the shaving head or over the sides and back of the shaving head. In another embodiment the cover may retreat inside one or more slots of the shaving head, at its open position (i.e. when the head is in contact with the skin). In any case, when in open position, the edge of the cover should be lower than the surface of the absorbent area and/or the shaving plane defined by the front shaving head components.

When the shaving head is not in touch with the skin, the cover may return back to its closed protective position, either manually, or through a mechanical spring effect or through an electromechanical action triggered by the skin contact sensor. The return to close position may be (essentially) instant. In other examples, the return to close position may have a hysteresis of more than 0.25, 0.5, 1, 2, 3, 4, 5, 10 or 20 seconds, e.g. to accommodate fast shaving strokes.

The shaving head 20 may comprise a heating element 23 configured to heat up a skin portion of the user in a shaving stroke. The heating element 23 may be adjacent to and/or around the absorbent area 21.

The heating element may exist in one or more edges or all around the absorbent area, in order to temporarily heat the skin to induce a quicker sweat or sebum release. In other words, heating may accelerate the sebum and sweat secretion. The heating element may be connected electrically and powered via a user input interface of shaver 10, the handle 30 and/or the health monitoring system. In other examples, the heating element may be activated by the skin contact/proximity sensor unit, when the shaver approaches or gets in contact with the skin.

The heating element 23 may comprise metal. Alternatively, or in addition, the heating element 23 may comprise ceramic and a semiconductor. Alternatively, or in addition, the heating element 23 may comprise thick film heaters. Alternatively, or in addition, the heating element 23 may comprise one or more polymer PTC heating elements.

For example, the heating element may be configured to be heated to a temperature ranging from 30 °C to 50 °C.

The shaving head 20 may comprise a contact sensor unit 24. The contact sensor unit 24 may comprise one or more contact sensors. The contact sensor unit 24 may be configured to output a contact signal indicative of the shaving head 20 being in skin contact.

The shaving head 20 may (also) comprise a proximity sensor unit 25. The proximity sensor unit 25 may comprise one or more proximity sensors. The proximity sensor unit 25 is configured to output a proximity signal indicative of the shaving head 20 being in proximity to and/or approaching the skin.

For example, one or more contact and/or proximity sensors may exist on any area of the shaving head, e.g. to actuate the cover opening and initiate the health test process. The one or more contact and/or proximity sensors may comprise a mechanical sensor, an optical sensor, a force sensor and/or a pressure sensor.

In another embodiment the contact sensor unit 24 may be located on the shaver handle and be activated through the force/pressure transfer of the skin to the shaving head and consequently to the shaving handle.

In examples the shaving head 20 may comprise the biosensor unit 40. The biosensor unit 40 may comprise one or more biosensors. The biosensor unit 40 (and the one or more biosensors therein) may be configured to analyze the body sample extracted from the user in the shaving stroke of the shaver 10, thereby generating an analysis result. The biosensor unit 40 may be integral to the shaving head 20 or part of a replaceable insert (e.g. also comprising the absorbent area 21 and the filter network 50) to the shaving head 20. For example, the biosensor unit may be located on the front, upper part of the shaving head, after the last blade.

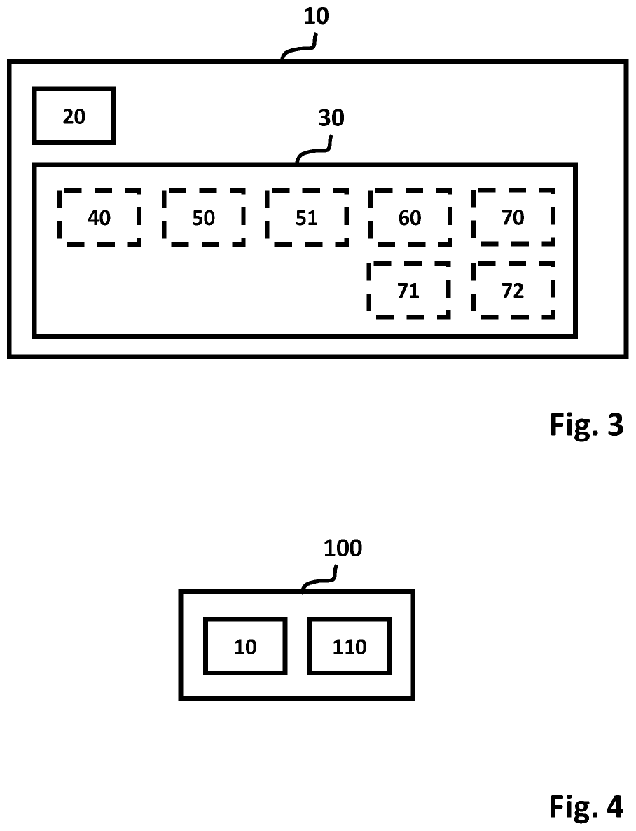

Furthermore, there is disclosed a shaver 10, as schematically illustrated in FIG. 3, comprising a handle 30 and the shaving head 20, the latter comprising the absorbent area 21.

The shaver 10 (i.e. not necessarily the shaving head of the shaver) may comprise the biosensor unit 40. The biosensor unit 40 may comprise the one or more biosensors. Again, the biosensor unit 40 may be configured to analyze the body sample extracted from the user in the shaving stroke of the shaver 10, thereby generating an analysis result.

In any embodiment, one or more biosensors may be configured to analyze one or more hormones such as e.g. endocrines like cortisol, testosterone and/or insulin. Alternatively, or in addition, one or more biosensors may be configured to analyze one or more antibodies such as e.g. immunologic like IgA, IgM and/or IgG. Alternatively, or in addition, one or more biosensors may be configured to analyze inflammatory such as e.g. one or more cytokines. Alternatively, or in addition, one or more biosensors may be configured to analyze one or more proteins such as e.g. enzymes. Alternatively, or in addition, one or more biosensors may be configured to analyze one or more metabolites such as e.g. vitamins. Alternatively, or in addition, one or more biosensors may be configured to analyze one or more cancer biomarkers. Alternatively, or in addition, one or more biosensors may be configured to analyze infectious and/or pathogen RNA.

A biosensor may be a bioelectrochemical sensor capable of analyzing the body sample such as e.g. bio fluids through enzyme-based electrocatalysis, in order to detect a biomarker, a virus or a bacterium and provide a signal. The biosensor may be connected to the filter network such as e.g. the microfluidic network collecting the body sample of the user.

One of the one or more biosensors may be an amperometric biosensor. Alternatively, or in addition, (another) one of the one or more biosensors may be an organic electrochemical transistor (OECT). Alternatively, or in addition, (another) one of the one or more biosensors may be an all-polymer micrometer-scale transistor biosensor. Alternatively, or in addition, (another) one of the one or more biosensors may be an optical biosensor. Alternatively, or in addition, (another) one of the one or more biosensors may be a thermal biosensor. Alternatively, or in addition, (another) one of the one or more biosensors may be a piezoelectric biosensor. An example low-cost polymer biosensor may be made of a n-type-conjugated polymer as the active material based on an NDI-T2 copolymer, which has a backbone comprising a highly electron-deficient naphthalene-1,4,5,8-tetracarboxylic diimide (NDI) repeat unit and an electron-rich unsubstituted bithiophene repeat unit (T2) called P-90. The side chains on the diimide unit may be a 90:10 randomly distributed ratio of polar glycol and nonpolar branched alkyl groups, where the ratio was optimized to ensure solubility of the copolymer in polar solvents. The polymer biosensor, as an electrolyte-gated transistor, is able to transduce ionic signals of biological origin into electronic ones, with high amplification. It may be comprised by an-type organic polymer semiconductor and/or an ion-to-electron converting device, i.e. the organic electrochemical transistor. The n-type polymer may incorporate hydrophilic side chains to enhance ion transport/injection, as well as to facilitate enzyme conjugation. The material is capable of accepting electrons of the enzymatic reaction and act as a series of redox centers capable of switching between the neutral and reduced state resulting to a fast, selective, and sensitive metabolite sensor. The advantages of the all-polymer biosensor may be that its functionalization is simple and the device design is elegant, obviating the need for mediators and a reference electrode. The voltage needed for the operation of the low-cost polymer biosensor may range from 200 mV to 1000 mV. The current needed for the operation of the biosensor may range from 0.5 μA to 3 μA. The power needed for the operation of the biosensor may range from 0.1 mW to 3 mW.

The biosensor transistor size may range from 10 μm to 200 μm. The biosensor thickness may range from 100 μm to 1000 μm. Biosensor may be directly powered by an electrical connection to the handle or powered by a wireless power transfer coil (WPT) connected to it.

In some embodiments, the handle 30 may comprise the biosensor unit 40. The biosensor unit 40 may be integral to the handle 30. In other examples, the biosensor unit 40 may be (part of) a replaceable insert to the handle 30.

In some embodiments, the shaving head 20 and the handle 30 may comprise the biosensor unit 40.

In some embodiments, the shaver 10 may comprise a module comprising the absorbent area 21, a filter network 50 and the biosensor unit 40 configured to analyze the body sample. For example, the module may be a replaceable insert.

For example, the module may be a fixed part of the shaving head 20, meant to be disposed with the shaving head 20 after one or more uses of the shaving head 20.

In other examples, the module may be a replaceable insert on the shaving head 20 with a respective gap over the blades area, meant to be replaced (only the insert) after one or more uses of the shaving head 20.

In yet other examples, the module may be a fixed extension of the handle 30, with a special adaptor so that a shaving head 20 with all the other necessary components may be inserted to after one or more uses.

In yet other examples, the module may be a replaceable insert on an extension of the handle 30, meant to be replaced along or independently of the shaving head 20.

In any case, the module may be either of analog type or electronic type. There may exist different interchangeable types of modules that may be used with the shaver 10, depending on their function and the health metric the user wants to detect during a specific shaving action. For example, the length of the module may range from 20 mm to 40 mm. For example, the width may range from 3 mm to 15 mm. For example, the thickness may range from 3 mm to 10 mm The module may be elevated relatively to the shaving head surface. The elevation height may range from 10 μm to 500 μm and, more preferably, from 50 μm to 150 μm. The elevation may be the same in all of its area or may be ascending with lower elevation at the edge located near the blade area and higher elevation at the opposite edge. The external surface of the module may be flat, concave or convex.

In case the module is electronic, an electrical connection may exist between the module and the (electronic) handle as well with the power source. The electrical connection may be wired or wireless. For example, the communication of the module with an electronic device 110 may be enabled by NFC.

The shaving head 20, the shaver 10, or the module may comprise a filter network 50 configured to filter and/or drive the body sample or parts thereof from the absorbent area 21 to the one or more sensors of the biosensor unit 40. The shaver 10 may comprise a filter element 51 configured to adjust and/or reset the filter network 50. For example, the filter network 50 may be a microfluidic network. For example, the filter network 50 may be a magnetically driven rotary microfilter configured to be adjusted and/or reset via an external magnetic field (e.g. resulting from an alternating magnetic field module).

For example, the filter network 50 may be located under the absorbent area 21 and between the absorbent area 21 and the biosensor unit 40. The filter network 50 may be connected to the absorbent area 21 and may receive the body sample or parts thereof (e.g. skin fluids and cells) collected by it.

In some embodiments, the filter network 50 may be limited to the shaving head 20.

In other embodiments, the filter network 50 may start from the absorbent area 21 located on the shaving head 20 and extend to the biosensor unit 40 which may be located in the handle 30.

The filter network such as e.g. the microfluidic network is capable of directing, mixing, separating and/or manipulating the collected body sample or parts thereof (e.g. biological molecules) to attain multiplexing, automation, and/or high throughput systems.

In the microfluidic network, the filtering of the biomolecules may be enabled by microfilters with fixed size depending on the biomolecules a user needs to detect and/or with magnetically driven rotating microfilters with different sizes located in the microfluidic network. The magnetically driven microfilters may be controlled by an external magnetic field generated by electromagnets and/or permanent magnets located around the filter network 50 (e.g. in the handle).

For example, by user control and setup with the relevant software, the magnetically rotating filters located in the microfluidic network may be set to allow the passage to one of the biosensors of one or more biomolecules and block the passage of other. Different setups may be chosen by the user at different times, for the same biosensor unit. The biomolecules filtered by each microfilter may be driven to different biosensors in order to perform many analyses at the same time or one after another.

The microfluidic network may comprise a plurality of microfluidic channels between the absorbent area 21 and the one or more biosensors of the biosensor unit 40. For example, the cross section of the microfluidic channels in the microfluidic network may be circular or rectangular. For example, the diameter of each microfluidic channel may range from 10 μm to 3 mm. The microfluidic channels may have the same or different diameters depending on their functions. The microfluidic channels may contain different microfilters to separate different molecules.

The material of the microfluidic network may be glass, silicon, polymer (PDMS) and/or paper based.

The filter element 51 may comprise or be an alternating magnetic field module. For example, the alternating direction magnetic field module may be located in an extension located at the internal top area of the handle 30 adjacent the microfluid network.

The magnetic field module may be a system of fixed electromagnetic microcoils, the magnetic field direction of which may vary periodically through a specific algorithm rotating the magnetically rotating microfluidic filters. The system may be arranged in a handle protrusion, with e.g. a length ranging from 2 cm to 4 cm. For example, the system of microcoils may contain from 1 to 10 microcoils that may be activated all at once or serially with a set frequency (e.g. depending on the testing protocol). Alternatively, or in addition, the magnetic field module may be a system of permanent micromagnets, which can rotate or translate mechanically inside the magnetic field module, varying the direction of the magnetic field exerted on the magnetically rotating microfilters. The super strong micromagnets may by of disc, block, rod shape or other custom shape. The type of the magnets may be any of NdFeb, SmCo, ceramic, Alnico and/or ferrite.

The shaver 10, the handle 30 and/or the module may comprise a communication module 60 configured to be communicatively coupled to an electronic device 110 of a health monitoring system 100.

Wireless connectivity may be provided for the biosensing unit and/or the module to interact with other devices (e.g. the handle 30 or the electronic device 110). For example, one of the following wireless protocols such as Wi-Fi, ANT+, Bluetooth Low Energy (BLE), IEEE 802.15.4 and/or NFC may be supported.

The shaver 10, the handle 30 and/or the module may comprise a DC power source. The electronic components of the shaver 10 may be powered by one or more disposable batteries, plug-in rechargeable batteries and/or wireless inductive charging module(s). For example, the power source and the associated electronic circuit may be configured to deliver up to 12 V, and more preferably, 3 V.

There is disclosed a health monitoring system 100, schematically illustrated in FIG. 4, comprising the shaver 10 (comprising the absorbent area 21 configured to be coupled to the biosensor unit 40 of the shaver 10) and an electronic device 110 configured to be communicatively coupled to the shaver 10. For example, the electronic device 110 may be a smartphone, a tablet, a smartwatch or a multi-purpose computer such as e.g. a PC.

The shaver 10, the handle 30 of the shaver 10, the module or the system 100 (e.g. the electronic device thereof) may comprise a user input interface 71. For example, the user input interface 71 may comprise control buttons, a keyboard, a mouse and/or a touchscreen.

For example, the control buttons may be located on the side of the handle 30. The control buttons may comprise one or more touch buttons. Alternatively, or in addition, the control buttons may comprise one or more switches. Alternatively, or in addition, the control buttons may comprise one or more rotation elements. Alternatively, or in addition, the control buttons may comprise one or more sliding elements.

Alternatively, or in addition, the shaver 10, the handle 30 of the shaver 10, the module or the system 100 (e.g. the electronic device thereof) may comprise a user output interface 72. For example, the user output interface 72 may be a display, a (touch)screen and/or a speaker.

For example, a display may exist on the handle 30 or on the back of the module. The display may be analog or electronic. For example, the electronic display may be an LCD, LED or electronic paper display. The display may show if a test is negative or positive by displaying a relevant symbol or value or text message once the analysis has been completed.

Alternatively, or in addition, the shaver 10, the handle 30 of the shaver 10, the module or the system 100 (e.g. the electronic device thereof) may comprise a computer system 70. For example, the computer system 70 may be (micro)controller.

For example, a microcontroller (MCU) may process and control all the sensors, circuits and functions of the shaver. It may contain a (flash) memory module. The microcontroller may be a conventional ultra-low power MCU suitable for wearable applications such as but not limited, a 16 or 32-bit-ARM MCU. Microcontroller may have a custom operating firmware.

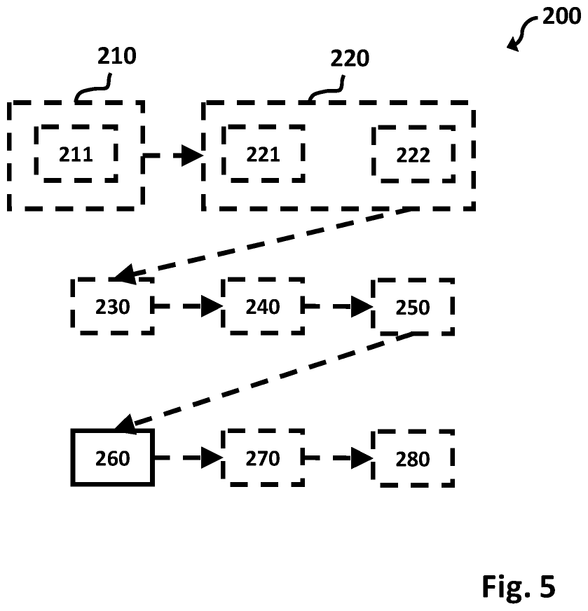

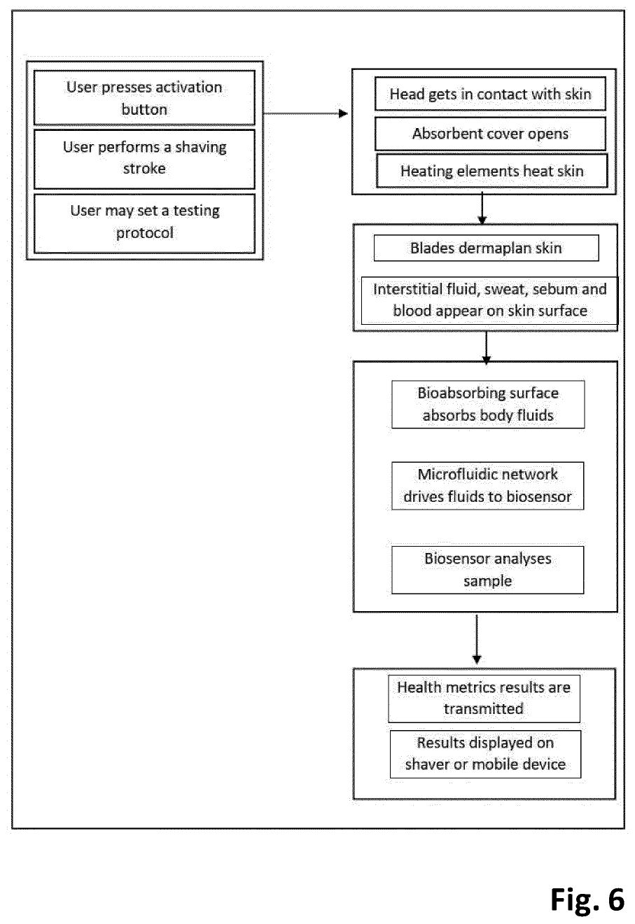

There is also disclosed a computer-implemented method 200 for health monitoring, comprising operating 260 a biosensor unit 40 in a shaver 10 to analyze a body sample extracted from a user in a shaving stroke of the shaver 10, thereby generating an analysis result. The method 200 is schematically illustrated in FIG. 5. Operating 260 the biosensor unit 40 may be based on a testing protocol. The testing protocol may be predetermined (at least at the point in time of carrying out the method 200).

The method 200 may comprise receiving 210, via a user input interface 71 of the shaver 10 and/or of an electronic device 110 communicatively coupled to the shaver 10, the testing protocol or one or more parameters thereof.

The method 200 may comprise reading 211 the testing protocol or one or more parameters thereof from a memory unit of the shaver 10 and/or of an electronic device 110 communicatively coupled to the shaver 10.

For example, and as e.g. indicated in FIG. 5, the method 200 may comprise receiving 210 user input and reading 211 a testing protocol corresponding to the user input from the memory unit.

The method 200 may comprise receiving 220 a start signal. Operating 260 the biosensor unit 40 to analyze the body sample may be started based on the start signal. For example, operating 260 the biosensor unit 40 may be started if the start signal is received. The start signal may be received 220 via a user input interface 71 of the shaver 10 and/or of an electronic device 110 communicatively coupled to the shaver 10. For example, and as in the example scenario given in FIG. 6, this may be realized by pressing an activation button.

The method 200 may comprise receiving 221 a contact signal from a contact sensor unit 24 in a shaving head 20 of the shaver 10, the contact signal indicative of the shaving head 20 being in skin contact. The start signal may be based on the contact signal. For example, the start signal may be the contact signal or depend on it.

The method 200 may comprise receiving 222 a proximity signal from a proximity sensor unit 25 in a shaving head 20 of the shaver 10, the proximity signal indicative of the shaving head 20 being in proximity to and/or approaching the skin. The start signal may be based on the proximity signal. For example, the start signal may be the proximity signal or depend on it.

For example, and as e.g. indicated in FIG. 5, the method 200 may comprise receiving 221 the contact signal and/or receiving 222 the proximity signal. Here the method 200 may further comprise receiving 220 the start signal that, for example, may be the contact signal and/or the proximity signal. In other examples, the start signal may be generated based on the contact signal and/or the proximity signal.

The method 200 may comprise operating 230 a cover actuator to open a cover 22 of an absorbent area 21 of a shaving head 20 of the shaver 10. The absorbent area 21 may be configured to receive the body sample when the cover 22 is open. The absorbent area 21 may be configured to do not receive the body sample when the cover 22 is closed. For example, operating 230 the cover actuator to open the cover 22 may be based on user input from a user input interface 71 of the shaver 10, of the module and/or of an electronic device 110 communicatively coupled to the shaver 10. In other examples, operating 230 the cover actuator to open the cover 22 may be based on the start signal, the contact signal and/or the proximity signal. For example, operating 230 the cover actuator to open the cover 22 may be carried out if the start signal, the contact signal and/or the proximity signal have been received.

The method 200 may comprise operating 240 a heating element 23 in a shaving head 20 of the shaver 10 to heat up a skin portion of the user to be shaved by the shaver 10. Operating 240 the heating element 23 to heat up the skin portion may be based on the testing protocol. For example, heating may be activated or increased, if sebum and/or sweat shall be analyzed. On the other hand, heating may be deactivated or decreased, if sebum and/or sweat shall not be analyzed. For example, operating 240 the heating element 23 to heat up the skin portion may be based on user input from a user input interface 71 of the shaver 10, of the module and/or of an electronic device 110 communicatively coupled to the shaver 10. In other examples, operating 240 the heating element 23 to heat up the skin portion may be based on the start signal, the contact and/or the proximity signal. For example, operating 240 the heating element 23 to heat up the skin portion may be carried out if the start signal, the contact signal and/or the proximity signal have been received.

For example, in contrast to the illustration in FIG. 5, steps 230 and 240—if both present—may be (essentially) simultaneous or step 240 may be carried out before step 230.

The method 200 may comprise operating 250 a filter element 51 to adjust a filter network 50, thereby filtering and/or driving the body sample or parts thereof to one or more sensors of the biosensor unit 40. Operating 250 the filter element 51 to adjust the filter network 50 may be based on the testing protocol. In so doing, the filter network 50 such as e.g. the microfluidic network may be adjusted (e.g. by adjusting the external magnetic field) so as to carry the analyses of the body sample or parts thereof as desired. For example, operating 250 the filter element 51 to adjust the filter network 50 may be based on user input from a user input interface 71 of the shaver 10, of the module and/or of an electronic device 110 communicatively coupled to the shaver 10. In other examples, operating 250 the filter element 51 to adjust the filter network 50 may be based on the start signal, the contact and/or the proximity signal. For example, operating 250 the filter element 51 to adjust the filter network 50 may be carried out after receiving the start signal, the contact and/or the proximity signal.

For example, in contrast to the illustration in FIG. 5, step 250—if present—may be carried out before any of the steps 230, 240—if present—or be (essentially) simultaneous thereto.

The method 200 may comprise transmitting the analysis result to a user output interface 72 of the shaver 10, of the module and/or of an electronic device 110 (e.g. a mobile device) communicatively coupled to the shaver 10. The method 200 may comprise visualizing the analysis result on the user output interface 72.

For example, the testing software may be a mobile device application. The testing software may comprise a user input interface 71. This may be the component that permits the user to set one or more testing protocols for the biosensor unit 40 or the (replaceable) module comprising the biosensor unit 40. It may be used to identify a specific module and enable the powering of the biosensor unit 40 and the wireless communication with it. It may also enable the activation of the magnetic field module and the use of specific microfilters depending on the testing protocol selected by the user.

For example, a communication algorithm may enable the communication of the NFC or another wireless communication method of the mobile device with the relevant chip embedded in the biosensing unit 40 and/or in the module. It may enable the wireless transmission and reception of data once the mobile device and the biosensing unit 40 (or the module) are at a close enough distance (e.g. 1 cm to 10 cm).

Visualizing the analysis result may be based on a visualization algorithm that may depend on user input.

The method 200 may comprise receiving an end signal. Operating 260 the biosensor unit 40 to analyze the body sample may be stopped based on the end signal. For example, the end signal may be received via a user input interface 71 of the shaver 10, of the module and/or of an electronic device 110 communicatively coupled to the shaver 10.

The method 200 may comprise receiving a not-in-contact signal from a contact sensor unit 24 in a shaving head 20 of the shaver 10, the not-in-contact signal indicative of the shaving head 20 not being in skin contact. The end signal may be based on the not-in-contact signal. For example, the end signal may be the not-in-contact signal or depend on it.

The method 200 may comprise receiving a not-in-proximity signal from a proximity sensor unit 25 in a shaving head 20 of the shaver 10, the not-in-proximity signal indicative of the shaving head 20 not being in proximity to and/or leaving the skin. The end signal may be based on the not-in-proximity signal. For example, the end signal may be the not-in-proximity signal or depend on it.

The method 200 may comprise operating 270 a cover actuator to close a cover 22 of an absorbent area 21 of a shaving head 20 of the shaver 10. For example, operating 270 the cover actuator to close the cover 22 may be based on user input from a user input interface 71 of the shaver 10 and/or of an electronic device 110 communicatively coupled to the shaver 10. In other examples, operating 270 the cover actuator to close the cover 22 may be based on the end signal, the not-in-contact signal and/or the not-in-proximity signal. For example, operating 270 the cover actuator to close the cover 22 may be carried out after receiving the end signal, the not-in-contact signal and/or the not-in-proximity signal. The cover actuator may be operated 270 to close the cover 22 after a delay of more than 0.25, 0.5, 1, 2, 3, 4, 5, 10 or 20 seconds. Delays may be beneficial in avoiding unintended closings of the cover in between shaving strokes.

The method 200 may comprise operating a heating element 23 in a shaving head 20 of the shaver 10 to stop heating up a skin portion of the user to be shaved by the shaver 10. For example, operating the heating element 23 to stop heating up the skin portion may be based on user input from a user input interface 71 of the shaver 10, of the module and/or of an electronic device 110 communicatively coupled to the shaver 10. In other examples, operating the heating element 23 to stop heating up the skin portion may be based on the end signal, the not-in-contact and/or the not-in-proximity signal. For example, operating the heating element 23 to stop heating up the skin portion may be carried out after receiving the end signal, the not-in-contact and/or the not-in-proximity signal. The heating element 23 may be operated to stop heating up the skin portion after a delay of more than 0.25, 0.5, 1, 2, 3, 4, 5, 10 or 20 seconds. Delays may be beneficial in avoiding unintended heating switches in between shaving strokes.

The method 200 may comprise operating 280 a filter element 51 to reset the filter network 50. Operating 280 the filter element 51 to reset the filter network 50 may be based on the testing protocol. For example, operating 280 the filter element 51 to reset the filter network 50 may be based on user input from a user input interface 71 of the shaver 10, of the module and/or of an electronic device 110 communicatively coupled to the shaver 10. In other examples, operating 280 the filter element 51 to reset the filter network 50 may be based on the end signal, the not-in-contact and/or the not-in-proximity signal. For example, operating 280 the filter element 51 to reset the filter network 50 may be carried out after receiving the end signal, the not-in-contact and/or the not-in-proximity signal. Resetting the filter network 50 may comprise getting rid of the body sample or parts thereof in the filter network 50. Resetting the filter network 50 may prevent the filter network from becoming encrusted and/or blocked.

There is disclosed a computer system 70 configured to execute the computer-implemented method 200 for health monitoring. The computer system 70 may be comprised by the shaver 10, the handle 30 of the shaver 10, the module and/or the system 100, see above. The computer system 70 such as e.g. a (micro)controller may comprise a processor and a RAM. The computer system 70 may comprise a non-volatile memory such as e.g. a flash memory.

There is disclosed a computer program configured to execute the computer-implemented method for health monitoring. The computer program may be interpretable and/or executable. The computer program may be loaded (also in part) as a bit or byte sequence into the RAM of the computer system 70.

There is disclosed a computer-readable medium or signal storing the computer program. For example, the medium may a volatile or non-volatile memory such as e.g. RAM, ROM, EPROM, HDD, SDD or the like.

Although the present subject-matter has been described above and is defined in the attached claims, it should be understood that the subject-matter may alternatively be defined in accordance with the following embodiments:
1. A shaving head (20) for a shaver (10) comprising:
   an absorbent area (21) configured to receive a body sample extracted from a user in a shaving stroke of the shaver (10);
wherein the absorbent area (21) is or may be coupled to a biosensor unit (40) of the shaver (10).
2. The shaving head (20) of embodiment 1, comprising:
   a filter network (50) configured to filter and/or drive the body sample or parts thereof from the absorbent area (21) to the one or more sensors of the biosensor unit (40).
3. The shaving head (20) of embodiment 2, wherein the filter network (50) is a microfluidic network.

4. The shaving head (20) of embodiment 2 or 3, wherein the filter network (50) is a magnetically driven rotary microfilter configured to be adjusted and/or reset via an external magnetic field.
5. The shaving head (20) of one of the preceding embodiments, comprising:
   a cover (22) configured to cover the absorbent area (21) in a first state and to uncover the absorbent area (21) in a second state.
6. The shaving head (20) of embodiment 5, comprising:
   a cover mechanism configured to close and/or open the cover (22).
7. The shaving head (20) of embodiment 6, wherein the cover mechanism comprises a cover actuator.
8 The shaving head (20) of one of the preceding embodiments, comprising:
   a heating element (23) configured to heat up a skin portion of the user in a shaving stroke.
9. The shaving head (20) of embodiment 8, wherein the heating element (23) is adjacent to and/or around the absorbent area (21).
10. The shaving head (20) of one of the preceding embodiments, comprising:
   a contact sensor unit (24) comprising one or more contact sensors.
11. The shaving head (20) of embodiment 10, wherein the contact sensor unit (24) is configured to output a contact signal indicative of the shaving head (20) being in skin contact.
12. The shaving head (20) of one of the preceding embodiments, comprising:
a proximity sensor unit (25) comprising one or more proximity sensors.
13. The shaving head (20) of embodiment 12, wherein the proximity sensor unit (25) is configured to output a proximity signal indicative of the shaving head (20) being in proximity to and/or approaching the skin.
14. The shaving head (20) of one of the preceding embodiments, comprising:
   the biosensor unit (40) comprising one or more biosensors;
wherein the biosensor unit (40) is configured to analyze the body sample extracted from the user in the shaving stroke of the shaver (10), thereby generating an analysis result.
15. The shaving head (20) of embodiment 14, wherein the biosensor unit (40) is integral to the shaving head (20) or a replaceable insert to the shaving head (20).
16. A shaver (10) comprising:
   a handle (30);
   the shaving head (20) according to one of the preceding embodiments.
17. The shaver (10) of embodiment 16, comprising:
   a biosensor unit (40) comprising one or more biosensors;
wherein the biosensor unit (40) is configured to analyze the body sample extracted from the user in the shaving stroke of the shaver (10), thereby generating an analysis result.
18. The shaving head (20) of embodiment 14 or 15 or the shaver (10) of embodiment 16, when dependent on embodiment 14 or 15, or of embodiment 17, wherein the one or more biosensors are configured to analyze one or more of:
   one or more hormones;
   one or more antibodies;
   one or more cytokines;
   one or more enzymes;
   one or more metabolites;
   one or more cancer biomarkers;
   infectious and/or pathogen RNA.
19. The shaver (10) of embodiment 17 or 18, wherein the handle (30) comprises the biosensor unit (40).
20. The shaver (10) of embodiment 19, wherein the biosensor unit (40) is integral to the handle (30) or a replaceable insert to the handle (30).
21. The shaver (10) of embodiment 17 or 18, wherein the shaving head (20) and the handle (30) comprise the biosensor unit (40).
22. The shaving head (20) of embodiment 14 or 15 or the shaver (10) of embodiment 16, when dependent on embodiment 14 or 15, or of one of the embodiments 17 to 21, comprising:
   a filter network (50) configured to filter and/or drive the body sample or parts thereof from the absorbent area (21) to the one or more sensors of the biosensor unit (40).
23. The shaving head (20) or shaver (10) of embodiment 22, wherein the filter network (50) is a microfluidic network.
24. The shaving head (20) or shaver (10) of embodiment 22 or 23, wherein the filter network (50) is a magnetically driven rotary microfilter configured to be adjusted and/or reset via an external magnetic field.
25. The shaver (10) of one of embodiments 19 to 24, wherein the shaver (10) comprises:
   a filter element (51) configured to adjust and/or reset the filter network (50).
26. The shaver (10) of one of the embodiments 19 to 25, wherein the filter element (51) comprises an alternating magnetic field module.
27. The shaver (10) of one of the embodiments 16 to 26, comprising:
   a communication module (60) configured to be communicatively coupled to an electronic device (110) of a health monitoring system (100).
28. A health monitoring system (100), comprising:
   the shaver (10) of one of the embodiments 16 to 27;
   an electronic device (110) configured to be communicatively coupled to the shaver (10).
29. The shaver (10) of one of the embodiments 16 to 27 or the system (100) of embodiment comprising:
   a user input interface (71).
30. The shaver (10) of one of the embodiments 16 to 27 or the system (100) of embodiment or 29, comprising:
   a user output interface (72).
31. The shaver (10) of one of the embodiments 16 to 27 or the system (100) of one of the embodiments 28 to 30, comprising:
   a computer system (70).
32. A computer-implemented method (200) for health monitoring, comprising:
   operating (260) a biosensor unit (40) in a shaver (10) to analyze a body sample extracted from a user in a shaving stroke of the shaver (10), thereby generating an analysis result.
33. The method (200) of embodiment 32, wherein operating (260) the biosensor unit (40) is based on a testing protocol.
34. The method (200) of embodiment 33, comprising:
   receiving (210), via a user input interface (71) of the shaver (10) and/or of an electronic device (110) communicatively coupled to the shaver (10), the testing protocol or one or more parameters thereof.
35. The method (200) of embodiment 33 or 34, comprising:
   reading (211) the testing protocol or one or more parameters thereof from a memory unit of the shaver (10) and/or of an electronic device (110) communicatively coupled to the shaver (10).
36. The method (200) of one of the embodiments 32 to 35, comprising:
   receiving (220) a start signal;
wherein operating (260) the biosensor unit (40) to analyze the body sample is started based on the start signal.
37. The method (200) of embodiment 36, wherein the start signal is received (220) via a user input interface (71) of the shaver (10) and/or of an electronic device (110) communicatively coupled to the shaver (10).
38. The method (200) of embodiment 36 or 37, comprising:
   receiving (221) a contact signal from a contact sensor unit (24) in a shaving head (20) of the shaver (10), the contact signal indicative of the shaving head (20) being in skin contact;
wherein the start signal is based on the contact signal.
39. The method (200) of one of the embodiments 36 to 38, comprising:
   receiving (222) a proximity signal from a proximity sensor unit (25) in a shaving head (20) of the shaver (10), the proximity signal indicative of the shaving head (20) being in proximity to and/or approaching the skin;
wherein the start signal is based on the proximity signal.
40. The method (200) of one of the embodiments 32 to 39, comprising:
   operating (230) a cover actuator to open a cover (22) of an absorbent area (21) of a shaving head (20) of the shaver (10);
wherein the absorbent area (21) is configured to receive the body sample when the cover (22) is open.
41. The method (200) of embodiment 40, wherein operating (230) the cover actuator to open the cover (22) is based on user input from a user input interface (71) of the shaver (10) and/or of an electronic device (110) communicatively coupled to the shaver (10).
42. The method (200) of embodiment 40 or 41, when dependent on embodiment 36, 38 and/or 39, wherein operating (230) the cover actuator to open the cover (22) is based on the start signal, the contact signal and/or the proximity signal.
43. The method (200) of one of the embodiments 32 to 42, comprising:
   operating (240) a heating element (23) in a shaving head (20) of the shaver (10) to heat up a skin portion of the user to be shaved by the shaver (10).
44. The method (200) of embodiment 43, when dependent on embodiment 33, wherein operating (240) the heating element (23) to heat up the skin portion is based on the testing protocol.
45. The method (200) of embodiment 43 or 44, wherein operating (240) the heating element (23) to heat up the skin portion is based on user input from a user input interface (71) of the shaver (10) and/or of an electronic device (110) communicatively coupled to the shaver (10).
46. The method (200) of one of the embodiments 43 to 45, when dependent on embodiment 36, 38 and/or 39, wherein operating (240) the heating element (23) to heat up the skin portion is based on the start signal, the contact and/or the proximity signal.
47. The method (200) of one of the embodiments 32 to 46, comprising:
   operating (250) a filter element (51) to adjust a filter network (50), thereby filtering and/or driving the body sample or parts thereof to one or more sensors of the biosensor unit (40).
48. The method (200) of embodiment 47, when dependent on embodiment 33, wherein operating (250) the filter element (51) to adjust the filter network (50) is based on the testing protocol.
49. The method (200) of embodiment 47 or 48, wherein operating (250) the filter element (51) to adjust the filter network (50) is based on user input from a user input interface (71) of the shaver (10) and/or of an electronic device (110) communicatively coupled to the shaver (10).
50. The method (200) of one of the embodiments 47 to 49, when dependent on embodiment 36, 38 and/or 39, wherein operating (250) the filter element (51) to adjust the filter network (50) is based on the start signal, the contact and/or the proximity signal.
51. The method (200) of one of the embodiments 32 to 50, comprising:
   operating (270) a cover actuator to close a cover (22) of an absorbent area (21) of a shaving head (20) of the shaver (10).
52. The method (200) of one of the embodiments 32 to 51, comprising:
   operating (280) a filter element (51) to reset a filter network (50).
53. A computer system (70) configured to execute the computer-implemented method (200) for health monitoring according to one of the embodiments 32 to 52.
54. A computer program configured to execute the computer-implemented method (200) for health monitoring according to one of the embodiments 32 to 52.
55. A computer-readable medium or signal storing the computer program of embodiment 54.

### REFERENCE NUMERALS

10 shaver
20 shaving head
21 absorbent area
22 cover
23 heating element
24 contact sensor unit
25 proximity sensor unit
30 handle
40 biosensor unit
50 filter network
51 filter element
60 communication module
70 computer system
71 user input interface
72 user output interface
100 health monitoring system
110 electronic device
200 computer-implemented method for health monitoring
210 receiving a testing protocol
211 reading the testing protocol
220 receiving a start signal
221 receiving a contact signal from a contact sensor unit
222 receiving a proximity signal from a proximity sensor unit
230 operating a cover actuator to open a cover of an absorbent area of a shaving head
240 operating a heating element in a shaving head
250 operating a filter element to adjust a filter network
260 operating a biosensor unit in a shaver to analyze a body sample
270 operating a cover actuator to close a cover of an absorbent area of a shaving head
280 operating a filter element to reset a filter network

1. A shaving head for a shaver comprising:
an absorbent area configured to receive a body sample extracted from a user in a shaving stroke of the shaver; wherein the absorbent area is coupled to a biosensor unit of the shaver; and
a filter network configured to filter and/or drive the body sample or parts thereof from the absorbent area to the one or more sensors of the biosensor unit.

2. The shaving head of claim 1, wherein the filter network is a microfluidic network.

3. The shaving head of claim 1, wherein the filter network is a magnetically driven rotary microfilter configured to be adjusted and/or reset via an external magnetic field.

4. The shaving head of claim 1, comprising:
a cover configured to cover the absorbent area in a first state and to uncover the absorbent area in a second state.

5-15. (canceled)

16. The shaving head of claim 4, comprising:
a cover mechanism configured to close and/or open the cover.

17. The shaving head of claim 16, wherein the cover mechanism comprises a cover actuator.

18. The shaving head of claim 1, comprising:
a proximity sensor unit comprising one or more proximity sensors, wherein the proximity sensor unit is configured to output a proximity signal indicative of the shaving head being in proximity to and/or approaching the skin.

19. The shaving head of claim 1, wherein the biosensor unit is integral to the shaving head or a replaceable insert to the shaving head.

20. The shaving head of claim 1, comprising:
a heating element configured to heat up a skin portion of the user in a shaving stroke.

21. The shaving head of claim 1, comprising:
a contact sensor unit comprising one or more contact sensors;
wherein the contact sensor unit is configured to output a contact signal indicative of the shaving head being in skin contact.

22. A shaver comprising:
a handle; and
the shaving head according to claim 1.

23. The shaver of claim 22, wherein the handle comprises:
a biosensor unit comprising one or more biosensors;
wherein the biosensor unit is configured to analyze the body sample extracted from the user in the shaving stroke of the shaver, thereby generating an analysis result.

24. The shaver of claim 22, comprising:
a filter element configured to adjust and/or reset the filter network.

25. The shaver of claim 24, wherein the filter element comprises an alternating magnetic field module.

26. A health monitoring system, comprising:
the shaver of one of the claims 22; and
an electronic device configured to be communicatively coupled to the shaver.

27. A computer-implemented method for health monitoring, comprising:
operating a biosensor unit in a shaver to analyze a body sample extracted from a user in a shaving stroke of the shaver, thereby generating an analysis result.

28. The method of claim 27, comprising:
operating a cover actuator to open a cover of an absorbent area of a shaving head of the shaver;
wherein the absorbent area is configured to receive the body sample when the cover is open.

29. The method of claim 27, comprising:
operating a heating element in a shaving head of the shaver to heat up a skin portion of the user to be shaved by the shaver.

30. The method of claim 27, comprising:
operating a filter element to adjust a filter network, thereby filtering and/or driving the body sample or parts thereof to one or more sensors of the biosensor unit; and/or
operating the filter element to reset the filter network.
